(19) United States
(12) Patent Application Publication
(10) Pub. No.: US 2019/0314298 A1
(43) Pub. Date: Oct. 17, 2019

(54) COMPOSITIONS FOR PREVENTING AND RELIEVING HANGOVER & LIVER DAMAGE WHICH OCCUR DUE TO ALCOHOL CONSUMPTION

(71) Applicant: Vasu Goyal, Montclair, CA (US)

(72) Inventors: Vasu Goyal, Montclair, CA (US); Ghanashyam Patel, Vadodara (IN)

(21) Appl. No.: 16/383,499

(22) Filed: Apr. 12, 2019

Related U.S. Application Data
(60) Provisional application No. 62/657,684, filed on Apr. 13, 2018.

(51) Int. Cl. A61K 31/12; A61K 36/9066; A61K 31/198; A61K 31/7004; A61K 9/00; A61P 1/16; A61K 36/889; A61K 36/185

(52) U.S. Cl. CPC A61K 31/12; A61K 36/9066; A61K 31/198; A61K 36/185; A61K 9/0095; A61P 1/16; A61K 36/889; A61K 31/7004

(57) ABSTRACT

The present invention is a beverage for relieving and preventing harmful effects of alcohol consumption such as hangover and liver cell damage, which includes Curcuma longa Rhizome extract, L-Ornithineamino acid, and Inositol as active ingredients. The curcumin extracts standardized to ≥95% pure curcuminoids having at least 12% in the form of water-dispersible micelle liquid or at least 10% in the form of nanosuspension. A method for beverage composition includes a defined amount of curcumin extract, L-Ornithine Inositol, and sugar syrup, wherein the curcumin extract, L-Ornithine and Inositol Powder added to the separately prepared sugar syrup under constant stirring. The present invention accelerates In vivo decomposition of alcohol after drinking, thereby helping to relieve and prevent harmful effects of alcohol such as hangover and liver damage.

### TECHNICAL FIELD

The present invention is a beverage composition for relieving and preventing harmful effects of alcohol consumption such as hangover and liver cell damage, which includes Turmeric (Curcuma longa) Rhizome extract, L-Ornithine HCL (an amino acid) and Inositol as active ingredients, prepared by using a unique process. The present invention accelerates In vivo decomposition of alcohol after drinking, thereby helping to relieve and prevent harmful effects of alcohol such as hangover and liver damage.

### BACKGROUND ART

Alcohol hangover refers to unpleasant physical and/or mental conditions experienced after drinking too much alcohol and objectively includes the following symptoms; decreased consciousness, nausea, vomiting, drowsiness, headache, decreased the ability to exercise, hematological variations, hormone changes, or the like.

Modern humans are exposed to a variety of stress during daily life, and the amount of stress that people experience continues to increase in modern society despite technological advancement.

Although many people attempt to overcome such stress in different ways such as exercise, sleeping, smoking, taking trips, or the like, the most common method in the modern society to relieve stress may be the drinking (alcohol).

The major reason for this is that, for modern people, drinking is not only a simple solution to overcoming stress but is also recognized as a culture obligation in a broad range of circumstances including, for example, improvement in business relationship through entertainment (or with a special service), bonding with co-workers and/or companions, family reunions, or the like.

In particular, as society becomes more complex and organized, frequent opportunities to drink are encountered, which in turn encourages heavy drinking and/or binge drinking. Since Koreans are obsessed with drinking and alcohol abuse due to Korean traditional drinking culture, Koreans often have hangovers and difficulty in recovering a normal condition or working the next day. Moreover, bad drinking habits continued for the long term can lead to health problems over time.

Ethanol absorbed into the body is mostly metabolized in the liver to form acetaldehyde, in turn being oxidized and discharged in the form of acetate. Oxidation from ethanol to acetaldehyde involves an enzyme, that is, alcohol dehydrogenase (ADH), while decomposition/oxidation of acetaldehyde generated from alcohol oxidation employs another enzyme named aldehyde dehydrogenase (ALDH).

According to normal alcohol metabolism, alcohol after drinking is absorbed in the stomach or the small intestine and passes through blood vessels into the liver. In liver cells, alcohol is oxidized into acetaldehyde by ADH and acetaldehyde is decomposed into acetate by ALDH, in turn, be decomposed into CO2 gas and water then discharged outside.

Although the exact cause of hangovers is not known, a number of suppositions such as toxicity of alcohol and alcohol metabolites (acetaldehyde, formaldehyde, acetone, etc), nutrient deficiency caused by nutrient absorption disorders, and so forth have been proposed. In recent years, formaldehyde in alcohol and metabolism of methanol contained in a small amount in alcohol has been indicated as causes of alcohol hangover(Jone A W. Pharmaco Toxicology. 60(3) 217-220). However, some documents have reported that hangover is caused by alcohol metabolites such as acetaldehyde, acetone(Tomita Y, Haseba T, Kurosu M, Watanabe T (1990), ArukoruKenkyutYakubutsulson. 25(2). 116-128; Tsukamoto S, Muto T. Nagoya T, Shimamura M, Saito T, Tainaka H. (1989), Alcohol alcohol. 24(2). 101-108).

Acetaldehyde generally includes Type 2 acetaldehyde to begin to be oxidized even when a concentration of in vivo acetaldehyde is low, and Type 1 acetaldehyde to begin to be oxidized only when a concentration of in vivo acetaldehyde is high. Type 2 acetaldehyde commonly generated in Asians is slowly oxidized, thus more frequently causing hangovers, as compared to Type 1 acetaldehyde generated in most westerners.

Because of social grounds of American drinking culture, consumers still need improved products for relieving hangovers and extensive studies and investigations into methods for relieving hangovers have been conducted in various applications. Variety of products are commercially available and/or have recently been introduced in the market.

However, although most products contain herbal materials and/or other compositions known to have bioactive efficiency, contents thereof are substantially too small. Also, since objective verification of the efficacy of final products manufactured in the art is still inadequate, the reliability of such products assessed by consumers who need effective hangover relief drinks is relatively low.

Accordingly, there is a strong need to develop an improved product having advantages of high reliability, decrease in social loss caused by heavy drinking, contribution to healthier lifestyles of individuals, or the like, by preparing the product from specific raw materials and/or compositions with advantageous effects for hangover relief and subjecting the prepared product to beverage tasting such that actual consumers directly drink the product and experience beneficial effects of the same, in turn assessing and verifying objective hangover relieving efficacy of the product.

There are several hangover remedies used around the world to reduce hangover. Most of these remedies are stand-alone compositions that are to be ingested before or after alcohol consumption. These include a composition of alcoholic neutralization ingredients, essentially made of a powder of Atractylis ovata Thunb, Poria cocoa Wolf, Alisma Plantago-Aquatica L, Pachma hoelen Rumph, Glycyrrhiza glabra L, Cinnamomum loureiru Nees, Thea Sinensis L, and Flos puerariae extracted from plants plus saccharine to be infused in cold water and consumed before or after the alcoholic drink to alleviate the hangover by promoting the hepatic metabolism of alcohol, according to U.S. Patent application No. 20040247704 (Chiang et al.) published on Dec. 9, 2004. In addition, a composition inhibiting pathological addiction to alcohol which contains a combination of different natural products is reported in U.S. Pat. No. 4,808,574 (Brekhman et al.); a method of altering the intoxicating effects of alcoholic beverages by the ingestion of activated charcoal prior to, along with/ or immediately following the consumption of alcohol is reported in U.S. Pat. No. 4,594,249 (Procter et al.) issued on Jun. 10, 1986; a mixture for lowering the concentration of alcohol in blood containing extracts of Pepino is reported in U.S. Pat. No. 6,713,091 (KIM) issued on Mar. 30, 2004; and natural teas from the raw material extract (or powder) of leaves, stems, or roots of alder and mountain ash which are optionally mixed with anti-dotal crude herb medicines including extract of Fructusligustic fruit and an extract of Radix puerariae in various ratios are claimed to be effective in curing hangover and reported in U.S. Pat. No. 5,968,520 (NAM et al.) issued on October 19, 1999. Patent CN107616499A discloses liver protection de-alcoholic composition and oral preparation. The patent composition comprises prepared from the following raw materials in parts by weight: 1-5 parts of an oyster extract, 1-150 parts of taurine, 0.1-5 parts of curcumin, 1-10 parts of methionine and 1-15 parts of Ornithine. Patent CN102764408B relates to the hangover formulation, specifically making a quick escape drinkers headache, nausea, and hangover formulation can recover in a short period of time, wherein: was prepared from the into, 400-800 mg lactose, starch 80-300 mg, 50-150 mg maltose, 50-100 mg of turmeric, curcumin 10-20 mg, 100-250 mg inositol, citric acid 30-70 mg, 10 to 40 mg niacin, vitamin C50-150 mg, vitamin E 4-8 mg, vitamin B65-15 mg, food flavors 30-70 mg, flavoring agents 50-250 mg. Compared with the prior art the present invention

Other publications in the art include WO 2005032569 (OHHIRA), which describes the use of the combination of turmeric and garlic as hangover remedial; KR 2004034761 (AN et al.), which describes a tonic beverage for reducing hangover comprising herbal medicines and additives.

Turmeric (Curcuma longa L.) Background and Benefits: Curcuma longa (Turmeric); U.S. Pat. No. 7,234,931 (LEE), which describes a food composition for relieving alcohol-induced hangover having 2 to 10% of Curcuma longa administered before or after alcohol consumption. KR 2000056670 (KANG), describing method of preparing alcoholic beverage using chrysanthemum; KR 2003075099 (CHOI et al.) describing a beverage for reducing hangover using Capsosiphonfulvescens extract; KR 2001045841 (SHIN), describing composition of alcoholic beverage made of medicinal herbs and causing no hangover and method thereof; KR 2003017796 (LIM), describing methods for manufacturing alcohol drinks causing no headache and hangover; JP 2006075059 (MIURA), describing alcoholic beverages containing vitamin B complex for imparting hangover-preventing effect; KR 2005017611 (MOON), describing a medicinal wine with medicinal herbs resulting in no hangover; KR 2005036061 (KIM), describing a liquor containing tourmaline which causes less hangover; KR 2005082800 (LEE), describing alcoholic drinks of pine mushroom that do not result in hangover; CN 1834219 (YANG), describing hangover-free mixed liquor with Hoveniadulcis fruit, Radix Puerariae, pectinase and yeast; KR 2003026382 (KIM), describing a method of manufacturing brandy containing propolis extract to reduce hangover; KR 2003021280 (KIM), describing a preparation of wine containing propolis extract to reduce hangover; KR 2001089930 (BAEK et al.), describing a preparation of pine needle wine by percolation with reduced hangover; KR 2003044698 (KIM), describing a functional alcohol with pine needle extract solution, mugwort extract solution and arrowroot extract solution having a hangover prevention effect; KR 2000026981 (SHIN), describing a herbal medicine wine eliminating hangover; U.S. Pat. No. 7,037,532 (FOXMAN), describing a hangover relief composition with six or seven homeopathic ingredients; and WO 2005123897 (LEE), describing a alcoholic beverage comprising polyphenols to prevent hangover.

Curcuma longa L (Turmeric) is an essential herb in India as well as South-East Asian countries. Diets rich in curcumin (a component of turmeric) have been considered the main reason for the lower rates of Alzheimer's disease among elderly East Indians, compared with elderly populations in Western countries. Turmeric is considered a cleansing herb for the whole body in Indian Ayurvedic medicine. It has also been used as a digestive aid and for the treatment of fever, infections, dysentery, arthritis, jaundice and other liver problems. Modern pharmacological studies have demonstrated that this herbal medicine exhibits antioxidant, antiprotozoal, nematocidal, anti-bacterial, anti-venom, anti-HIV, and antitumor activities.

Turmeric is extremely safe. It has been used in large quantities as food with no adverse reactions. However, persons with symptoms from gallstones should avoid turmeric. Turmeric's potential anti-clotting effect might cause problems for those with clotting disorders. An unusually large amount of turmeric consumption may result in stomach upset.

Turmeric comprises different curcuminoids, the most important of which are illustrated in the table below. Curcumin has been shown to have the following effects: (1) protect against free radical damage (strong antioxidant); (2) reduce inflammation (by reducing histamine levels and possibly by increasing production of natural cortisone by the adrenal glands); (3) protect the liver from a number of toxic compounds; (4) reduce platelets from clumping together, which in turn, improves circulation and helps protect against atherosclerosis; and(5) cancer-preventing effects which may be due to its powerful antioxidant activity in the body.

Turmeric products such as capsules containing standardized powder, tablets, and tincture are available as health products for the prophylaxis or treatment of several physiological malfunctions. Due to its non-toxic characteristics, except for a few drug interactions, turmeric (or UKON in Japanese) is considered to be a safe nutraceutical. There are two main UKON crops in Japan, AKI-UKON (Fall crop) and HARU-UKON (Spring crop). AKI-UKON is the crop of choice because it has the highest concentration of curcumin and, more generally, of curcuminoids.

The table below illustrates the curcumin and its derivatives:

TABLE
Curcumin and derivatives from Curcuma longa L. with biological activities

| Compounds | Chemical structure | Activity |
|---|---|---|
| Curcumin | 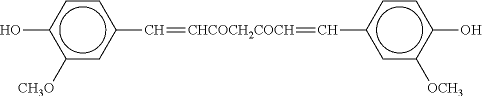 | anti-bacteria Leishmania amazonensis anti-HIV antioxidant anti-inflammatory anti-tumor |
| Ar-turmerone | 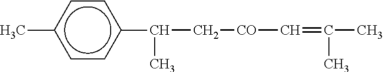 | snakebite |
| Methylcurcumin | 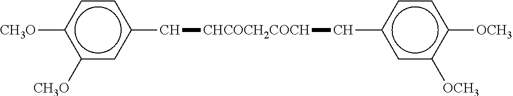 | L. amazonensis |
| Demethoxy curcumin | 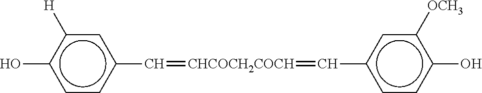 | antioxidant |
| Bisdemethoxy curcumin | 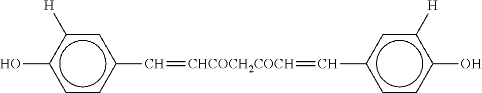 | antioxidant |
| Sodium curcuminate | 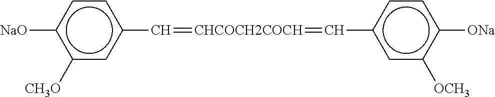 | anti-inflammatory |

Ornithine HCL Background and Benefits: L-Ornithine is an amino acid that is primarily used in the urea cycle, which eliminates excess nitrogen from the body. It isn't an essential amino acid in humans, meaning that it can be synthesized in the body. Ammonia (NH3) is a waste product of cellular metabolism, which becomes toxic if allowed to accumulate. L-Ornithine is a catalyst in the process that converts ammonia into urea, which can be eliminated through urine.

The liver uses the enzyme arginase and the amino acid L-arginine to synthesize urea and L-Ornithine as part of the urea cycle. E. coli in the intestines also synthesizes L-Ornithinefrom the amino acid L-glutamate. L-Ornithineserves as the precursor for various other important compounds such as glutamic acid, proline, and citrulline. The highest concentrations of L-Ornithine are found in connective tissue such as the skin.

Animal protein is the best dietary source of L-Ornithine, like other amino acids. This primarily includes meat, eggs, and dairy products. L-Ornithine is also present in nuts such as coconut, peanuts, and walnuts. Grains such as oats and wheat contain L-Ornithine, as do beans such as carob and soybeans. L-Ornithine is often prepared for use in health supplements as L-OrnithineHCL, which is the hydrochloride salt of L-Ornithine.

The primary use of L-Ornithine in health supplements is to support athletic performance. It is also used to support liver functioning, healthy wound recovery, and stress management.

Inositol Background and Benefits: Inositol, known chemically as cyclohexane-1,2,3,4,5,6-hexol, has the chemical formula. The most common of inositol's nine forms, or stereoisomers, in nature, is cis-1,2,3,5-trans-4,6-cyclohexanehexol, commonly known as myo-inositol.

Myo-inositol performs many essential functions for eukaryotic cells, which includes all plants and animals. It serves as the structural basis for cell messengers, primarily inositol phosphates. It is also a component of phosphatidylinositol and other phospholipids, which are used to construct cell membranes. Plants commonly use inositol hexaphosphate, commonly known as phytic acid, as a means of storing phosphates.

Significant quantities of pure inositol don't exist in nature. However, the associated lipids and phosphates of inositol are found in many foods, especially oranges and cantaloupes. Phytic acid is a phosphate of inositol that is most common in cereal with high bran content. It is also found in seeds, nuts, and beans, although it is not digestible by humans without cooking. Lecithins are also a widely available source of inositol that's relatively easy to digest in raw form.

Uses of Inositol: Relief from panic disorders is one of the common uses of inositol. It may also help with the symptoms of polycystic ovary syndrome (PCOS), which is a common condition among obese women. Additional problems that inositol may benefit include OCD.

Curcumin can be used as a hangover remedy. For example, it can be consumed before or after heavy drinking, normally in a concentrated form such as a pill. After thorough research, the present inventors have now found that Curcumin in micelle liquid form along with L-Ornithine and Inositol can be used to prepare beverage that effectively prevents harmful effects of alcohol consumption such as hangover and liver damage.

As far as is known to the inventors, this was never done before. Many reasons can be invoked to explain this. First, as mentioned above, Curcumin is highly insoluble in water, and its bioavailability during digestion is not good enough to produce the desired effect as an anti-hangover agent. Apart from this, alcohol after entering into bloodstream exerts detrimental effects on Liver and brain cells. Turmeric extract alone in any form is not good enough to prevent such damage; hence it should be supported by other actives.

The person of skill in the art would, therefore, have expected that combination of Curcumin in micelle liquid medium in combination with L-Ornithine and Inositol could be able to attenuate prevention and remedial support to Liver and brain cell damage.

### SUMMARY OF THE INVENTION

The present invention relates to a beverage for relieving and preventing harmful effects of alcohol consumption such as hangover comprising the combination of 3 actives ingredients such as Curcumin derived from Turmeric (Curcuma longa) Rhizome, L-Ornithineamino acid and Inositol. The of 3 actives ingredients such as Curcumin derived from Turmeric (Curcuma longa) Rhizome, L-Ornithineamino acid and Inositol are mixed with separately prepared sugar syrup to complete the beverage composition.

The invention also relates to the use of 3 actives mentioned above in the manufacture of anti-hangover beverage.

In embodiments of the invention, the curcumin may be in the form of an extract. In specific embodiments, the extract may be an ethanolic extract. In embodiments, the extract may be in the form of micelle prepared in a liquid medium to attain stable water dispersion or in the form submicron suspension in the presence of polysorbate 80 or lecithin as solubilizer/surfactant modifiers

In embodiments, curcumin would preferably be a nanoparticle suspension of wherein the nanoparticles would be of an averages size of less than about 2,000 nm to about 100 nm. Preferably the nanoparticles would have an average particle size (d50) of from about 1,600 nm to about 400 nm and most preferably about 1,400 nm to about 900 nm. Preferably the d90 will be less than about 5,000 nm and more preferably less than about 4,400 nm.

In embodiments, the Curcumin micelle liquid or curcumin nanosuspension provides about 12 mg to 45 mg of >95% pure curcuminoids per 70 ml of the beverage.

In embodiments, the beverage may contain pharmaceutically approved additives along with flavoring agents for better consumer compliance.

The main advantage of the present invention is that the present invention provides a beverage for relieving and preventing the harmful effects of alcohol consumption.

Yet another advantage of the present invention is that the present invention accelerates in vivo decomposition of alcohol after drinking.

Yet another advantage of the present invention is that the present invention reduces the harmful effects of alcohol consumption such as hangover and liver damage.

Yet another advantage of the present invention is that the efficacy of the herbal ingredient of the present invention is very high as compared to other similar invention.

Yet another advantage of the present invention is that due to more bioavailability of the herbal ingredient, a beverage made using herbal ingredient of the present invention produces quick and optimum hangover and liver damage preventing and relieving effect.

Yet another advantage of the present invention is that the present invention helps in protection of the liver and brain cells.

Yet another advantage of the present invention is that the present invention contains an adequate number of electrolytes which replenishes their loss during excessive urination caused by alcohol ingestion.

Yet another advantage of the present invention is that the present invention increases the energy level of the body.

Further objectives, advantages and features of the present invention will become apparent from the detailed description provided herein below, in which various embodiments of the disclosed invention are illustrated by way of example and appropriate reference to accompanying drawings.

### BRIEF DESCRIPTION OF THE ACCOMPANYING DRAWING

The aspects mentioned above and other features of the present invention will be explained in the following description, taken in conjunction with the accompanying drawings, wherein:

FIG. 1 illustrates results of self-awareness efficacy assessment for relief in a hangover.

FIG. 2 illustrates assessment results efficacy per individual hangover symptom.

### OBJECTS OF THE INVENTION

The main objective of the invention to provide a beverage composition for relieving and preventing a hangover.

Another objective of the present invention is to reduce the damage to the liver and reduce hangover due to alcohol composition.

Further objectives, advantages and features of the present invention will become apparent from the detailed description provided herein below, in which various embodiments of the disclosed invention are illustrated by way of example and appropriate reference to accompanying drawings.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments for those skilled in the art to practice the invention.

### Definition

The terms “a” or “an,” as used herein, are defined as one or as more than one. The term “plurality,” as used herein, is defined as two or as more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended. The term “comprising” is used interchangeably used by the terms “having” or “containing.”

Reference throughout this document to “one embodiment”, “certain embodiments”, “an embodiment”, “another embodiment”, and “yet another embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics are combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C.” An exception to this definition will occur only when a combination of elements, functions, steps or acts are in some way inherently mutually exclusive.

As used herein, the term “one or more” generally refers to, but not limited to, singular as well as the plural form of the term.

The drawings featured in the figures are to illustrate certain convenient embodiments of the present invention and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

The present invention is a beverage for relieving and preventing harmful effects of alcohol consumption such as hangover and liver cell damage, which includes Turmeric (Curcuma longa) Rhizome extract, L-Ornithine HCL (an amino acid) and Inositol as active ingredients, prepared using unique process.

The present inventors have thus taken various In-vivo trials to establish this hypothesis and found that the beverages made with Curcumin, L-Ornithine and Inositol is highly effective in preventing hangover and liver cell damage.

The present invention relates to a beverage for relieving and preventing a hangover, including the foregoing composition, more specifically, a method for preparation of the composition for relieving and preventing hangover according to the present invention may include the following:

In the present embodiment of the present invention the beverage composition prepared by first preparing sugar syrup which is made by the following process. 5.1587% of sugar is to be dissolved in purified water 92.03% of its total volume. All water-soluble additives such as Citric acid 0.5503%, Sodium citrate 0.2119%, Potassium citrate 0.3284%, Sodium benzoate 0.0894%, Potassium sorbate 0.0894%, Stevia leaf extract 0.0179%, Xanthan gum 0.0825% are to be added one by one under stirring condition. Curcumin micelle liquid (with at least 6% curcuminoids) 0.4952% or Curcumin nanosuspension (with at least 10% curcuminoids), L-Ornithine powder 0.4328% and Inositol powder 0.1197% are to be added at the end to prepare beverage liquid. Separately, 0.02889% and 0.1032% of two natural flavor solutions are to be added in prepared beverage liquid under stirring and mix well to attain homogenous ready to drink beverage.

In another embodiment of the present invention the beverage composition prepared by first preparing sugar syrup which is made by the following process. 5.1587% of sugar is to be dissolved in purified water 92.03% of its total volume. All water-soluble additives such as Citric acid 0.5503%, Sodium citrate 0.2119%, Potassium citrate 0.3284%, Sodium benzoate 0.0894%, Potassium sorbate 0.0894%, Stevia leaf extract 0.0179%, Xanthan gum 0.0825% are to be added one by one under stirring condition. Curcumin micelle liquid (with at least 6% curcuminoids) 0.4952% or Curcumin nanosuspension (with at least 10% curcuminoids), Amala (Indian gooseberry) extract 0.2139%, Coconut water concentrate 0.2139% L-Ornithine powder 0.4278% and Inositol powder 0.1197% is to be added at the end to prepare beverage liquid. Separately, 0.02889% and 0.1032% of two natural flavor solutions are to be added in prepared beverage liquid under stirring and mix well to attain homogenous ready to drink beverage.

In order to attain hangover relief (and prevention) efficacy as described above, a total amount of the beverage composition comprising major active ingredients such as Curcumin(with at least 6% or 10% curcuminoids), L-Ornithine and Inositol powder proposed according to the present invention, for an adult weighing 70 kg average. The prepared composition may be taken without particular limitations. The number of taking the composition may be limited as per the consumption limit of additives such as polyols, preservatives, sweetening agents, electrolytes, etc.

Using the active combination according to the present invention, in addition to beverages (or drinks), various formulations for relieving hangovers such as liquid preparations (gels, extract), pills, powder, tablets, capsules, or the like may be manufactured for oral administration.

In another embodiment of the present invention the Curcumin may be in the form of an extract. Such an extract has the advantage of allowing the easy introduction of the active curcuminoids of the turmeric in the beverage. The extract may be prepared by any method of extraction known in the art. The extract powder may be further purified to achieve >95% pure curcuminoids. This specific turmeric extracts further processed with this extract a micelle liquid form and allows it to provide stable water dispersion which essentially improves its bioavailability by many folds in the human intestine during digestion.

In embodiments, Curcumin extract further processed to nanosuspension form with surface modifiers preferably Lecithin and allows it to provide stable water dispersion which essentially improves its bioavailability by many folds in the human intestine during digestion.

In embodiments, the Curcumin micelle liquid provides about 12 mg of ≥95% pure curcuminoids per 70 ml of the beverage.

In embodiments, the Curcumin nanosuspension provides about 45 mg of ≥95% pure curcuminoids per 70 ml of the beverage.

In the further embodiment of the present invention the L-Ornithine and Inositol may be in the form of a powder. These powders may be pharmaceutical grade from commerce.

The beverage apart from 3 actives may contain natural or artificial sweetener, a polyol such as Sorbitol or Erythritol, Citric acid as an acidity regulator, Sodium citrate and Potassium citrate as electrolytes, permitted preservatives, thickener, natural and natural identical flavor and water as a carrier.

In another embodiment the beverage apart from 5 actives may contain natural or artificial sweetener, a polyol such as Sorbitol or Erythritol, Citric acid as an acidity regulator, Sodium citrate and Potassium citrate as electrolytes, permitted preservatives, thickener, natural and natural identical flavor and water as a carrier.

### DESCRIPTION OF THE DRAWINGS

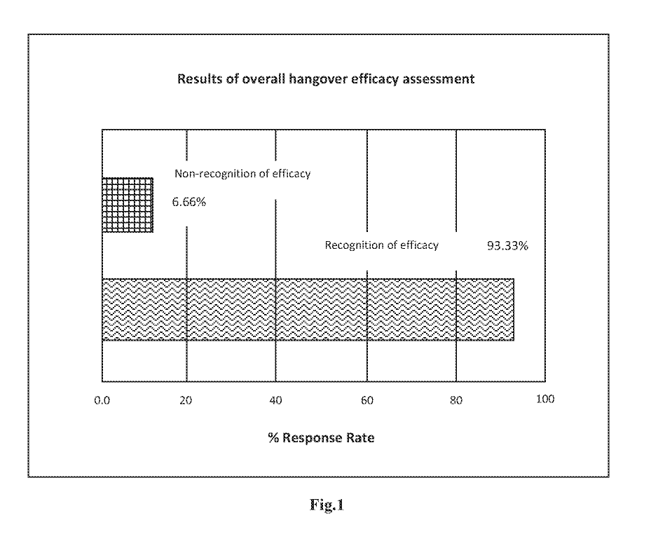

FIG. 1 illustrates assessment results of improvement in overall hangover extents after taking a composition for relieving and preventing hangover according to the present invention before drinking alcohol, based on results of self-awareness efficacy tests in relation to hangover relief using the composition for relieving and preventing hangover according to the present invention.

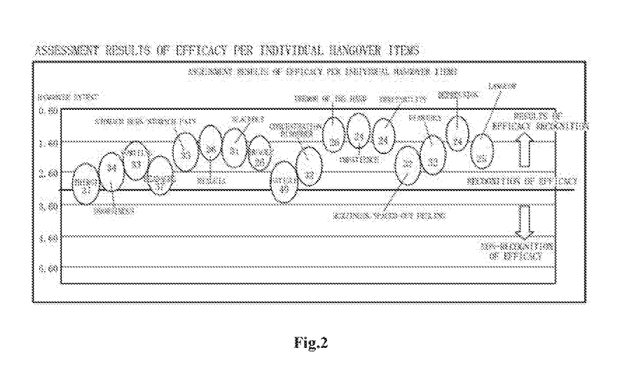

FIG. 2 illustrates the assessment results of improvement in different hangover symptoms after taking a beverage for relieving and preventing hangover according to the present invention before drinking alcohol.

### PRACTICAL EMBODIMENTS OF THE PRESENT INVENTION WILL BE DESCRIBED IN DETAIL ACCORDING TO THE FOLLOWING EXAMPLES

### Example 1

Preparation of Turmeric (Curcuma longa L) Rhizome extract (with at least 6% curcuminoids) in liquid form or nanosuspension form

Curcumin derived from Turmeric(Curcuma longa L) Rhizome extract (with at least 6% curcuminoids), L-Ornithine and Inositol powder are procured from commercial supply.

### Example 2

Preparation of a Beverage Composition for Relieving Hangover before and after Drinking

Beverage for the present invention may be prepared by first preparing Sugar syrup which is made by the following process. 5.1587% of sugar is to be dissolved in purified water 92.03% of its total volume. All water-soluble additives such as Citric acid 0.5503%, Sodium citrate 0.2119%, Potassium citrate 0.3284%, Sodium benzoate 0.0894%, Potassium sorbate 0.0894%, Stevia leaf extract 0.0179%, Xanthan gum 0.0825% are to be added one by one under stirring condition. Curcumin micelle liquid (with at least 6% curcuminoids) 0.4952% or curcumin nanosuspension (at least 10% curcuminoids), L-Ornithine powder 0.4328% and Inositol powder 0.1197% are to be added at the end to prepare beverage liquid. Separately, 0.02889% and 0.1032% of two natural flavor solutions are to be added in prepared beverage liquid under stirring and mix well to attain homogenous ready to drink beverage. After that, the obtained product was filled in a suitable container, labeled and packaged, thereby producing a hangover relief beverage (drink).

### Experimental Example 1

Clinical Study Design & Duration:

The present study is a double-blind, in-house, randomized, two-arm parallel, placebo-controlled study for evaluation of the efficacy and safety of investigational beverage product in people with a hangover. The person selected given the willingness to undergo alcohol drinking aged between 30 to 50 years were selected for the study based on inclusion criteria. The total duration of the study for a patient was not exceeding 10 days. Enrolled Thirty (30) Healthy male volunteers were divided into two groups, 15 volunteers in each group.

Group A (Test Group) was given health drink of SHOT-X of 70 ml 30 minutes after the last drink. [All volunteers took nearly 140 ml (1.8-2 ml/kg) of whiskey (42.8% w/v) of the same brand and batch. The duration of the drink was 1 hour.]

Group B (Placebo Group) was given 70 ml of PLACEBO drink containing no actives. 30 minutes after the last drink. [All volunteers took nearly 140 ml (1.8-2 ml/kg) of whiskey (42.8% w/v) of the same brand and batch. The duration of the drink was 1 hour.]

Criteria for the Assessment:

The patients were evaluated for primary endpoints & secondary endpoints parameters.

Primary Endpoints:
1. ALT, AST, and ALP: Baseline, 2 and 10 hours of formulation
2. ALDH (Acetaldehyde Dehydrogenase): Baseline, after 0, 2 hours and 10 hours of the formulation
3. Blood Aldehyde: Baseline, after 0, 2 hours and 10 hours of the formulation
4. Blood Alcohol: Baseline, after 0, 2 hours and 10 hours of the formulation

Secondary Endpoints:
1. Questionnaire to ask to understand the level of hangover
2. Walk test on a drawn single line on the floor (15 meters) to understand the level of hangover
3. Incidence and rate of adverse events, if any

Statistical Analysis:

Data analysis was performed using SAS®statistical software version 9.1.3 at 5% level of significance. Values were expressed in Mean ±SD. P <0.05 was considered as statistically significant p<0.01 was considered as statistically Highly significant and p<0.001 was considered as Very highly significant.

Results& Discussion:

A total number of 30 subjects were involved in this study and all patients had completed the study. No drop-out was observed.

Effect on Alanine aminotransferase (ALT) level:

TABLE 1
Effect on Alanine aminotransferase (ALT) level in different groups

| Groups | Baseline | | 2 Hours | | 10 hours | |
|---|---|---|---|---|---|---|
| Group A | 47.22 ± | 9.87 | 40.20 ± | 11.11* | 38.07 ± | 9.42 |
| Group B | 41.53 ± | 10.43 | 38.80 ± | 8.55 | 40.80 ± | 8.74 |

Paired t-test was applied within a group where n=15 for each group; Values were expressed in Mean ±SEM.Different significant levels were marked as *p<0.05, #p<0.01, ##p<0.001.The results output for Alanine aminotransferase (ALT) showed that ‘p’ value obtained for Baseline vs. 2 hours for Group A was found to be 0.0130. This suggests that a statistically significant change has been observed from Baseline to 2 hours values for Group A whereas no statistically significant change has been noticed in Group B. There was no statistically significant change from Baseline to 10 hours values for Group A and Group B values of Alanine Aminotransferase. (Table 1).Normally, the AST level in our blood is low. If the liver is damaged AST level is a rise in blood. Data of Table 1 directs that Test drink Shot-X was able to make a significant reduction in AST level which shows the liver protective property of the Shot-X.

Effect on Aspartate aminotransferase (AST) level:

TABLE 2
Effect on Aspartate aminotransferase (AST) level in different groups

| Groups | Baseline | | 2 Hours | | 10 hours | |
|---|---|---|---|---|---|---|
| Group A | 25.05 ± | 5.30 | 24.87 ± | 9.80 | 24.20 ± | 7.56 |
| Group B | 24.73 ± | 10.56 | 22.87 ± | 9.26 | 24.40 ± | 8.08 |

Paired t-test was applied within a group where n=15 for each group; Values were expressed in Mean ±SEM. Different significant levels were marked as *p<0.05, #p<0.01, ##p<0.001.The null difference was observed in Aspartate aminotransferase (AST) level of both the groups at baseline, 2 hours & 10 hours values. It indicates no effect of test drink on this parameter. (Table 2) .

Effect on Alkaline phosphatase (ALP) level:

TABLE 3
Effect on Alkaline phosphatase (ALP) level in different groups

| Groups | Baseline | | 2 Hours | | 10 hours | |
|---|---|---|---|---|---|---|
| Group A | 93.39 ± | 17.15 | 20.67 ± | 7.93^{##} | 36.60 ± | 6.20^{##} |
| Group B | 84.73 ± | 23.67 | 61.20 ± | 7.06^{#} | 62.00 ± | 5.50^{#} |

Paired t-test was applied within a group where n=15 for each group; Values were expressed in Mean ±SEM. Different significant levels were marked as *p<0.05, #p<0.01, ##p<0.001.A significant reduction was observed in Alkaline phosphatase (ALP) level of both groups in comparison to baseline value however Group A showed highly significant change as compared to Group B. Significant increase in ALP level at 10 hours in comparison of 2 hours value shows signs of recovery and effect of test drink. Similar recovery was also observed in the Placebo group but a lesser proportion. (Table 3)

on ALDH (Acetaldehyde Dehydrogenase) & Blood Aldehyde:

TABLE 4
Effect on ALDH (Acetaldehyde Dehydrogenase) level in different
groups

| Groups | Baseline | | 2 Hours | | 10 hours | |
|---|---|---|---|---|---|---|
| Group A | 62.47 ± | 3.27 | 118.83 ± | 12.34^{##} | 88.82 ± | 5.40^{##} |
| Group B | 62.93 ± | 4.57 | 88.17 ± | 7.13^{##} | 74.37 ± | 5.11^{##} |

Paired t-test was applied within a group where n=15 for each group; Values were expressed in Mean ±SEM. Different significant levels were marked as *p<0.05, #p<0.01, ##p<0.001.After consumption of alcohol Acetaldehyde dehydrogenase (ALDH) level is increased and over the period of time metabolism and excretion of alcohol take place its level down. Level of ALDH may vary based on the frequency of alcohol consumption, the volume of alcohol, metabolism rate of an individual, age & sex, etc. Statistically, a significant rise was observed from Baseline to 2 hours values for Group A and Group B, and at 10 hours these values were significantly reduced near to baseline value.(Table 4)

TABLE 5
Effect on Blood Aldehyde level in different groups

| Groups | Baseline | | 2 Hours | | 10 hours | |
|---|---|---|---|---|---|---|
| Group A | 1.470 ± | 0.36 | 3.155 ± | 0.15^{##} | 1.379 ± | 0.34^{##} |
| Group B | 1.578 ± | 0.26 | 3.57 ± | 0.25^{##} | 2.873 ± | 0.24^{#} |

Paired t-test was applied within a group where n=15 for each group; Values were expressed in Mean ±SEM. Different significant levels were marked as *p<0.05, #p<0.01, ##p<0.001.The increase in ALDH levels directly lead to a decrease in Blood Aldehyde levels and thus hangover effect is reduced. Elevated blood acetaldehyde causes facial flushing, severe headache, palpitations, tachycardia, hypertension, respiratory distress, nausea and vomiting which are common symptoms of a hangover. A significant rise in ALDH level in the test group was directly correlated with a decrease in blood aldehyde level which indicates the effect of Shot-X on a hangover. This clear correlation was found with the clinical data in Placebo group as we have observed 14 subjects to have a hangover in Placebo group and also their Blood aldehyde levels were found on the higher side, i.e. their ALDH values were on the lower side which leads to a mild hangover. This is due to the active compound of Shot-X. It is augmenting the normal physiological phase-II detoxification by increasing the level of ALDH thereby causing a significant reduction in blood aldehyde levels which explains the absence of hangover in the group of subjects who were dosed with Shot-X in comparison to Placebo.

Effect on Blood Alcohol level:

TABLE 6
Effect on Blood Alcohol level in different groups

| Groups | Baseline | | 2 Hours | | 10 hours | |
|---|---|---|---|---|---|---|
| Group A | 0.0021 ± | 0.0008 | 0.4053 ± | 0.0608^{##} | 0.0018 ± | 0.0015* |
| Group B | 0.0024 ± | 0.0011 | 0.4180 ± | 0.0517^{##} | 0.0033 ± | 0.0016* |

Paired t-test was applied within a group where n=15 for each group; Values were expressed in Mean ±SEM. Different significant levels were marked as *p<0.05, #p<0.01, ##p<0.001.Data of Table 6 indicates that no significant difference was observed in alcohol levels of Group A & Group B at baseline. However, it was clearly evidenced that after 10 hours mean blood alcohol levels were much better controlled by Group A in comparison to Group B.

Effect on hangover symptoms & walk test:

Highly significant effect was noticed during the evaluation of test drink Shot-X on hangover symptoms. In Group A, Hangover symptoms like Feeling thirsty, Stomach ache, Feeling nausea, Heart palpitations, Loss of appetite and Feeling tired were not observed in any subjects. Only one subject felt headache & dizziness in Group A. Overall 93.33% subjects were found no hangover wherein placebo group, i.e. in Group B, 86.66% subjects were reported with mild to moderate hangover during walk test.

Conclusion:

From the present data, it can be concluded that Shot-X is effectively able to increase the level of Acetaldehyde Dehydrogenase (ALDH) and thereby decreasing the Blood Aldehyde levels which will alleviate the symptoms of a hangover. Shot-X is also helpful in reducing liver enzymes which shows its hepatoprotective activity. As far as its safety concern, there were no adverse events were observed during the clinical trial. It proves that Shot-X-liquid curcumin-based oral drink is safer and efficient to diminish hangover symptoms caused due to alcohol intoxication.

### Experimental Example 2

Hangover Self-Awareness Efficacy Verification Test

A test procedure for hangover self-awareness efficacy was conceived and practically conducted on the basis of hangover assessment question items disclosed in a foreign document (Pittler, M. H. et al. CMAJ* 2003:169:1269-1273, Canadian medical association or its licensors), and an assessment procedure and data for hangover extents which are applied to “Research report regarding development of hangover relieving efficacy protocol” by the Korean food and drug administration (KFDA).

Age and gender distributions of participants for the present test are shown in TABLE 7.

### Description of the Tables

TABLE 7 illustrates the distribution of all 15 male Test panelists for the Hangover Self-Awareness Efficacy Verification Test.

| Age wise distribution | Male |
|---|---|
| 29 years old or less | 4 |
| 30 to 34 years old | 6 |
| 35 to 39 years old | 3 |
| 40 to 44 years old | 2 |
| Total | 15 |

In respective test groups, all male participants had an average drinking capacity of 3 bottles.

After providing 70 ml (1 bottle) of the beverage composition for relieving and preventing hangover prepared in Example 2 according to the present invention to each of 15 men, the participants were allowed to sufficiently drink within individual drinking capacities. The following day after drinking, a questionnaire (that is, checklist) regarding self-awareness for hangover relieving efficacy was given to each participant to investigate hangover states, and the participants replied to questions on the checklist, to thereby conduct question assessment of self-awareness efficacy for hangover relief.

Ingredients of the composition for relieving and preventing hangover developed according to the present invention, which was used in the previous assessment, are shown in TABLE 8 (see Example 2). To evaluate self-awareness efficacy for hangover relief, the checklist used in the present invention is shown in TABLE 9.

TABLET 8: Table 8 illustrates the Composition of the beverage for relieving and preventing hangover taken by test panelists.

Composition of the beverage for relieving and preventing hangover taken by test panelists was as followed.

| Ingredients | % | 70 ml | UOM |
|---|---|---|---|
| Curcumin (6% Curcuminoids) liquid extract* | 0.4952 | 360.00 | Mg |
| L-Ornithine HCL | 0.4328 | 314.60 | Mg |
| Inositol | 0.1197 | 87.00 | Mg |
| Sugar, Granulated | 5.1587 | 3750.00 | Mg |
| Stevia leaf extract | 0.0179 | 13.00 | Mg |
| Citric acid | 0.5503 | 400.00 | Mg |
| Potassium Sorbate | 0.0894 | 65.00 | Mg |
| Potassium citrate | 0.3284 | 238.70 | Mg |
| Sodium Benzoate | 0.0894 | 65.00 | Mg |
| Sodium Citrate | 0.2119 | 154.00 | Mg |
| Natural Orange Cream Flavour | 0.2889 | 210.00 | Mg |
| Natural Orange Sweet Juicy Flavour | 0.1032 | 75.00 | Mg |
| Purified Water | Q.S. | Q.S. | |

*Curcumin nanosuspension (10% curcumiods): 216 mg

TABLE 9 illustrates the Checklist for assessment of self-awareness efficacy for hangover relief

Checklist for assessment of self-awareness efficacy for hangover relief

| | Hangover | | | | | |
|---|---|---|---|---|---|---|
| | relieving | Hangover extent No - - - - - - - - - - - > | | | | |
| | efficacy | Serious | | | | |
| Symptoms | (Yes, No) | 1 | 2 | 3 | 4 | 5 |
| Thirst | | | | | | |
| Drowsiness | | | | | | |
| Vomiting/ | | | | | | |
| nausea/feeling | | | | | | |
| sick | | | | | | |
| Headache | | | | | | |
| Stomach burn/ | | | | | | |
| abdominal pain/ | | | | | | |
| stomach pain | | | | | | |
| Myalgia | | | | | | |
| Short time | | | | | | |
| blackout | | | | | | |
| Hunger | | | | | | |
| Fatigue/ | | | | | | |
| tiredness | | | | | | |
| Concentration | | | | | | |
| decrease/ | | | | | | |
| concentration | | | | | | |
| disorder | | | | | | |
| Tremor of | | | | | | |
| hands and feet | | | | | | |
| Impatience/ | | | | | | |
| anger | | | | | | |
| Irritability | | | | | | |
| Fuzziness/ | | | | | | |
| spaced-out | | | | | | |
| feeling | | | | | | |
| Diarrhoea | | | | | | |
| Depression | | | | | | |
| Languor | | | | | | |
| Overall | | | | | | |
| hangover | | | | | | |
| symptoms | | | | | | |

Assessment standards for scores given by participants
(1) No hangover: no difficulty (or trouble) in daily life
(2) Little hangover: There is scarcely any difficulty (or trouble) in daily life
(3) Being usual: a hangover is less than ordinary times and no difficulty (or trouble) in daily life
(4) General hangover: difficulty (or trouble) in daily life
(5) Heavy hangover: a hangover is serious about having difficulty (or trouble) in daily life

As a result of self-awareness efficacy assessment, it was found that improvement effects (decrease in hangover symptoms) were recorded in most question items regarding such hangover symptoms when the participants took the composition for relieving and preventing hangover according to the present invention. Specifically, with regard to important symptom items showing high response rate (75% or more) such as fatigue, headache, thirst, stomach burn/stomach pain, drowsiness, vomiting, etc., among hangover assessment items, the participants reported good effects in a range of at least 70% and up to 85%. Therefore, it can be understood that the inventive composition for relieving and preventing hangover exhibits remarkably improved effects in relieving a hangover. With regard to other items such as tremor of the hand, impatience, irritability, languor, Myalgia, etc., the inventive composition also exhibited improved effects. However, since the foregoing items have relatively decreased response rate by the participants, compared to other items, it was considered that symptoms in these items are not major hangover symptoms. Detailed test results are shown in TABLE 4.

TABLE 10
illustrates the detailed results of Table7, Table 8 and Table 9.

| | | | | | | Stomach | | | Black |
|---|---|---|---|---|---|---|---|---|---|
| | Section | Thirst | Drowsiness | Vomiting | Headache | burn/pain | Fatigue | Myalgia | out |
| Total | Avg. extent | 2.66 | 2.28 | 1.97 | 2.47 | 1.58 | 2.57 | 1.25 | 1.55 |
| 15 | of cognitive | | | | | | | | |
| | symptoms | | | | | | | | |
| | Avg. | 90% | 80% | 80% | 81% | 77% | 81% | 78% | 79% |
| | Response | | | | | | | | |
| | rate % | | | | | | | | |
| | | | | Tremor | | | | | |
| | | | Concentration | of the | | | | | |
| | Section | Hunger | disorder | hand | Impatience | Irritability | Fuzziness | Diarrhea | Languor |
| Total | Avg. | 1.90 | 1.99 | 1.07 | 1.52 | 1.86 | 2.06 | 1.91 | 1.32 |
| 15 | extent of | | | | | | | | |
| | cognitive | | | | | | | | |
| | symptoms | | | | | | | | |
| | Avg. | 77% | 81% | 75% | 82% | 76% | 83% | 83% | 77% |
| | Response | | | | | | | | |
| | rate % | | | | | | | | |

Moreover, as a result of confirming whether the composition for relieving and preventing hangover according to the present invention has effects of relieving or improving overall hangover symptoms to all 15 male participants who participated in the present test recognized hangover relieving efficacy of the inventive composition. Consequently, it can be identified that products developed according to the present invention may have excellent effects in relieving hangovers.

As apparent from the foregoing, the beverage composition according to the present invention may exhibit excellent effects in hangover relief and prevention, as demonstrated from self-awareness efficacy tests.

Although the preferred embodiments of the present invention have been disclosed for illustrative purposes, those skilled in the art will appreciate that various modifications, additions, and substitutions are possible, without departing from the scope and spirit of the invention as disclosed in the accompanying claims.

Further objectives, advantages and features of the present invention will become apparent from the detailed description provided herein below, in which various embodiments of the disclosed present invention are illustrated by way of example and appropriate reference to accompanying drawings. Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A beverage composition said composition comprising:
0.4952% curcumin extract, wherein the curcumin extract contains at least 6% curcuminoids or
0.31% curcumin extract in the form of nanosuspension contains at least 10% curcuminoids;
0.4328% L-Ornithine; and
0.1197% Inositol;
Thereby the curcumin extract, L-Ornithine and Inositol mixed with separately prepared sugar syrup to complete said beverage composition.

2. The beverage composition as claimed in 1, wherein the L-Ornithine and Inositol used in powder form.

3. The composition as claimed in claim 1, wherein 0.02889% and 0.1032% of two natural flavor solution optionally added separately to the prepared beverage composition under constant stirring.

4. The beverage composition as claimed in claim 1, wherein the curcumin extract derived from Turmeric (Curcuma longa) Rhizome standardized to >95% pure curcuminoids having at least 12% in the form of micelle liquid.

5. The beverage composition as claimed in claim 1, wherein the curcumin extract is treated with an amphiphilic liquid substance which make the curcumin extract in the form of micelle liquid form.

6. The beverage composition as claimed in claim 1, wherein the curcumin extract is subjected to particle size reduction technique with surface modifiers which make the curcumin extract in the form nanosuspension.

7. The beverage composition as claimed in claim 1, wherein said beverage composition is in liquid form.

8. The beverage composition as claimed in claim 1, wherein the curcumin extract, L-Ornithine and Inositol are the Active Ingredients of the beverage composition.

9. The composition as claimed in claim 1, wherein 0.2931% of Amala (Indian gooseberry) extract and 0.2931% of Coconut water concentrate are used in the beverage composition.

10. The beverage composition as claimed in claim 1, wherein said beverage composition used for relieving and preventing liver cell damage and hangover.

11. The beverage composition as claimed in claim 1, wherein the method for preparing sugar syrup comprising:
Adding5.2% sugar, wherein 5.2% of sugar dissolved in purified water 92.03% of its total volume; and
adding water-soluble additive such as Citric acid 0.5503%, Sodium citrate 0.2119%, Potassium citrate 0.3284%, Sodium benzoate 0.0894%, Potassium sorbate 0.0894%, Stevia leaf extract 0.0179%, Xanthan gum 0.0825% one by one under stirring condition.

12. A method for beverage composition, wherein said method comprises:
0.4952% curcumin extract, wherein the curcumin extract contains at least 6% curcuminoids or
0.31% curcumin extract in the form nanosuspension contains at least 10% curcuminoids;
0.4328% L-Ornithine;
0.1197% Inositol; and
Sugar syrup,
Wherein the curcumin extract, L-Ornithine and Inositol Powder added at the end to prepare sugar syrup under constant stirring.

13. The method for beverage composition as claimed in claim 10, wherein 0.02889% and 0.1032% natural flavor solution added separately to the prepared beverage composition under constant stirring.

14. The method for beverage composition as claimed in 10, wherein the L-Ornithine and Inositol used in powder form.
